(19) United States
(12) Patent Application Publication
(10) Pub. No.: US 2010/0312006 A1
(43) Pub. Date: Dec. 9, 2010

(54) PRODUCTION OF LEVULINIC ACID AND LEVULINATE ESTERS FROM BIOMASS

(75) Inventors: Michael A. Lake, Mount Pleasant, SC (US); Steven W. Burton, Cary, NC (US); William Craig Fuller, Raleigh, NC (US); Robert Sasser, Lumberton, TX (US); Mathias E. Lindstrom, Raleigh, NC (US); Jeffrey T. Wheless, Louisburg, NC (US)

Correspondence Address: MEADWESTVACO CORPORATION;ATTN: IP LEGAL DEPARTMENT, 1021 MAIN CAMPUS DRIVE, RALEIGH, NC, 27606, US

(73) Assignee: MEADWESTVACO CORPORATION, Richmond, VA (US)

(21) Appl. No.: 12/793,806

(22) Filed: Jun. 4, 2010

Related U.S. Application Data
(60) Provisional application No. 61/185,290, filed on Jun. 9, 2009.

(51) Int. Cl. C07C 51/00; C07C 69/716; C07C 59/185

(52) U.S. Cl. 560/174; 562/513; 562/577

(57) ABSTRACT

A process for producing levulinic acid and its esters from biomass is disclosed comprising: (i) feed preparation module characterized by subjecting biomass to a high-temperature refining treatment; (ii) hydrolysis reaction module that facilitates the hydrolysis of biomass to its respective sugars and their subsequent transformation to levulinic acid, formic acid, furfural, and char as well as facilitates the separation of lignin-based char by-product; (iii) product separation and recovery module utilizing a solvent extraction technique such as using furfural by-product as extracting solvent; and (iv) optionally, conversion of levulinic acid to levulinate ester. When desired, the disclosed process may be integrated into existing pulp mills.

This non-provisional application relies on the filing date of provisional U.S. Application Ser. No. 61/185,290 filed on Jun. 9, 2009, having been filed within twelve (12) months thereof, which is incorporated herein by reference, and priority thereto is claimed under 35 USC §1.19(e).

### BACKGROUND OF THE DISCLOSURE

Levulinic acid has been recognized in various applications. It is a starting material for the production of a variety of industrial and pharmaceutical compounds such as resins, plasticizers, herbicides, and specialty chemicals. Nonetheless, its commercial significance has been limited due in part to its high production cost. Several methods have been reported for preparing levulinic acids. However, these synthetic methods often require high-cost raw materials and/or provide low synthetic yields.

Research effort has been spent developing an economically viable and environmentally safe process for producing levulinic acid, particularly from an inexpensive and renewable feedstock such as biomass. U.S. Pat. No. 5,859,263 describes a process for producing levulinic acid by extruding a mixture of starch, water and mineral acid in a screw extruder at a temperature of 80° C.-150° C. Then, the levulinic acid is isolated from the reaction product mixture by a series of steps: filtration, steam distillation, condensation, and finally centrifugation. U.S. Pat. No. 5,608,105 discloses a process of producing levulinic acid and formic acid from carbohydrate-containing raw materials using two reactors in which the temperature, reaction time, and acid content are closely controlled. The raw materials are supplied to a first reactor and hydrolyzed at between 210° C.-230° C. in the presence of mineral acid to produce hydroxymethylfurfural along with other reaction intermediates, which are then conveyed into a second reactor. The resulting hydroxymethylfurfural is hydrolyzed further at 195° C.-215° C. in the presence of mineral acid in the second reactor to produce levulinic acid, furfural, formic acid, and other by-products. To facilitate the separation of levulinic acid from the product mixture, the process conditions of the second reactor are adjusted such that furfural and formic acid are vaporized and externally condensed, whereas the levulinic acid is concentrated at the bottom of the reactor. Once the concentration of the levulinic acid is sufficiently high, a stream containing levulinic acid is removed from the steady-state reaction mix in the reactor. The concentration of levulinic acid in the collected stream is further increased by evaporation, as well as separating sulfuric acid from the stream by neutralization with sodium hydroxide to participate out sodium sulfate solid. U.S. Pat. No. 6,054,611 discusses a production of levulinic acid from sugars that are, in turn, generated from a strong acid hydrolysis of biomass.

Oxygenated fuel additives are chemicals capable of raising the oxygen content of the fuels, thereby allowing the fuels to be burn more completely and consequently reducing harmful tailpipe emissions from motor vehicles. Methyl tertiary-butyl ether (MTBE) is an example of a widely used oxygenated fuel additive. However, there has been increasing environmental concerns regarding the detection of MTBE in surface and ground water. The ester derivative of levulinate acid has been identified as a renewable, environmental friendly alternative for MTBE as oxygenated fuel additives for diesel fuels, gasoline fuels, biofuels, and blends thereof.

The known methods of preparing levulinate ester by esterifying levulinic acid with alcohol have several drawbacks. First, the esterification process must proceed to near completion in order to minimize the level of unreacted levunic acid. Levulinate esters for the fuel additive applications must contain a minimum level of acid in order to prevent corrosion of the fuel container and the engine parts. Second, the esterification process is a reversible reaction that generates water as a by-product. Water forms azeotrope with low-molecular weight alcohols; therefore, it is difficult to separate water out of the reaction mixture and force the esterification process to a high yield. Third, the esterification of levulinic acid must be done at high pressures to accelerate a liquid-liquid reaction; otherwise, the esterification reaction is limited by the low boiling point of the alcohol. Several alternative methods of preparing levulinic ester have been reported.

U.S. Pat. No. 7,153,996 describes a process for preparing a mixture comprising levulinic esters and formic esters from biomass and olefin, wherein the olefin is used to facilitate the separation of levulinic acid from other reaction products. Biomass is acidic hydrolyzed to provide a mixture of levulinic acid, formic acid and furfural. Then, the aqueous product mixture is reacted with at least one olefin, optionally in the presence of a second acid catalyst, to produce an aqueous phase and an organic phase containing levulinic esters and formic esters. Finally, the organic phase containing desired products is separated from the aqueous phase. U.S. Pat. No. 7,378,549 teaches a reactive extraction of levulinic acid from an aqueous mixture having levulinic acid. The aqueous mixture is contacted with a liquid esterifying alcohol that substantially water-immiscible and comprises at least four carbon atoms, at esterification conditions in the presence of a catalyst to form a levulinate ester, wherein the amount of the alcohol is such that the alcohol extracts the levulinate ester from the aqueous mixture. This provides an aqueous phase having the catalyst and a reduced levulinic acid content and an organic phase having the alcohol and the levulinate ester.

There is still a need for a process for producing levulinic acid and its esters from biomass that is economically viable, energy efficient and environmental friendly, yet providing the desired products with enhanced purity and product yields.

Further, it is desirable that such process may be integrated into an existing manufacturing facility to further minimize energy consumption, production cost, and transportation cost.

### SUMMARY OF THE DISCLOSURE

A process for producing levulinic acid and its esters from biomass is disclosed that is economical and energy efficient, as well as provides enhanced product yield and purity. The disclosed process comprises: (i) feed preparation module characterized by subjecting biomass to a high-temperature refining treatment; (ii) hydrolysis reaction module that facilitates the hydrolysis of biomass to its respective sugars and their subsequent transformation to levulinic acid, formic acid, furfural, and char as well as facilitates the separation of lignin-based char by-product; (iii) product separation and recovery module utilizing a solvent extraction technique such as using furfural by-product as extracting solvent; and (iv) optionally, conversion of levulinic acid to levulinate ester. Levulinic acid may be converted to levulinate ester by first esterification of the acid with glycerol to provide glycerol levulinate and subsequently transesterification the resulting glycerol ester with mono-alcohol. The disclosed process may be integrated into an existing manufacturing facility to further minimize energy consumption, production cost, and transportation cost. When desired, the disclosed process may be integrated into existing pulp mills.

### BRIEF DESCRIPTION OF THE DRAWINGS

FIG. 1 is a schematic diagram showing one embodiment of the process of producing levulinic acid-based compounds from biomass of the present disclosure, wherein the process is integrated into an existing pulp mill.

### DETAILED DESCRIPTION OF THE DISCLOSURE

The present disclosure now will be described more fully hereinafter, but not all embodiments of the disclosure are necessarily shown. While the disclosure has been described with reference to exemplary embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof.

A process of producing levulinic acid-based compounds from biomass of the present disclosure comprises:
(i) a feed preparation module characterized by subjecting biomass to a high-temperature refining treatment to generate fiber pulps;
(ii) a hydrolysis reaction module wherein the generated fiber pulps are hydrolyzed to provide a product mixture including levulinic acid; and
(iii) a product separation and recovery module comprising a solvent extraction process to isolate the levulinic acid from the product mixture.

When desired, the disclosed process may further include a levulinic acid conversion module, wherein the generated levulinic acid is converted to its derivatives. These derivatives may include, but are not limited to, esters, amides, alcohols, acid anhydrides, and salts.

In one embodiment of the present disclosure, the process of producing levulinic acid-based compounds from biomass comprises:
(i) a feed preparation module characterized by subjecting biomass to a high-temperature refining treatment to generate fiber pulps;
(ii) a hydrolysis reaction module wherein the generated fiber pulps are hydrolyzed to provide a product mixture including levulinic acid;
(iii) a product separation and recovery module comprising a solvent extraction process to isolate the levulinic acid from the product mixture; and
(iv) an esterification module converting the levulinic acid to levulinate ester.

In one embodiment, the disclosed process may be integrated into existing manufacturing facilities in order to further improve operating cost and energy efficiency. Examples of such manufacturing facilities include, but are not limited to, pulp mills, ethanol manufacturing plants, biodiesel manufacturing plants, recycle plants, and utilities plants with cogeneration.

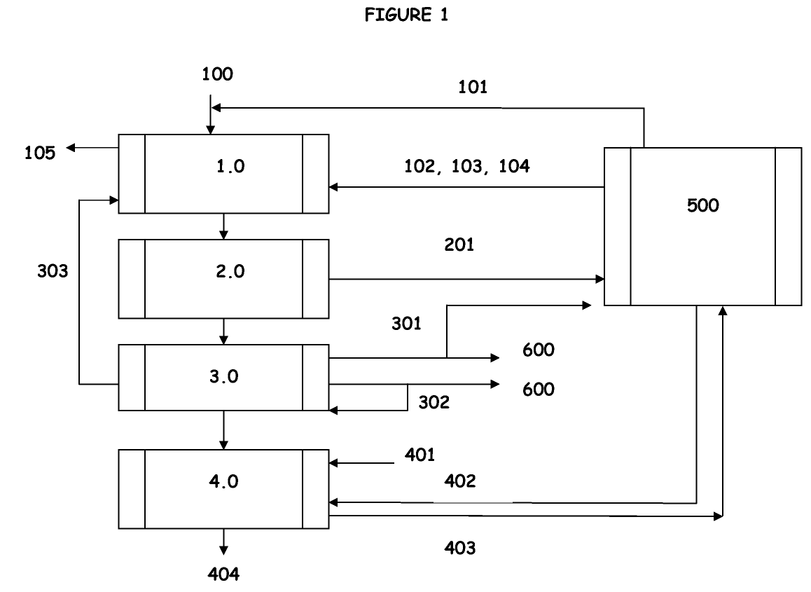

FIG. 1 shows a schematic diagram of one embodiment of the disclosed process of producing levulinic acid-based compounds from biomass, wherein the disclosed process is integrated into a pulp mill host (500). Biomass (100) is fed into the Feed Preparation Module (Module 1.0), wherein biomass (100) is converted into fiber pulps and the volatile organic materials in the biomass are vaporized. These vaporized volatile organic materials (105) may include, for example, turpentine and methanol. Other by-products from the pulp mill host (101) may be fed into Module 1.0 along with the biomass (100). Examples of such by-products (101) include, but are not limited to, woodyard waste, sludge, trimming, chips, and combinations thereof. Furthermore, the pulp mill host (500) may function as a source for water (102), steam (103), and electric energy (104) for Module 1.0.

Subsequently, the generated fiber pulps are subjected to the Hydrolysis Reaction Module (Module 2.0), wherein the hydrolysis reaction of fiber pulps takes place and levulinic acid is produced as one of the hydrolysis products along with other products including formic acid, furfural, acid catalyst such as sulfuric acid, and char by-product (201). Under the high temperature and pressurized conditions of Module 2.0, the char (201) may be isolated from the hydrolyzed mixture stream and the recovered chars may be used as energy sources for the production of levulinic acid from biomass, as well as for the host manufacturing facility (500) itself.

In the Product Separation and Recovery Module (Module 3.0), the produced levulinic acid is then separated from the hydrolysis product mixture. For example, the levulinic acid may be isolated from the mixture using furfural as an extracting solvent to separate the desired product from the hydrolysis product mixture. The isolated formic acid (301) may be used as a raw material for the manufacturing host such as host pulp mill (500) or sold to customers (600) after purification. The isolated furfural solvent (302) may be recycled back to the Module 3.0 and reused as the extracting solvent or sold to customers (600) after purification. The recovered acid catalyst such as sulfuric acid (303) may be recycled back to the Feed Preparation Module (Module 1.0).

In the Esterification Module (Module 4.0) of FIG. 1, the isolated levulinic acid from Module 3.0 may be esterified with a selected alcohol (401) and then the resulting levulinic ester may be transesterified with methanol (402) fed from the host manufacturing site such as host pulp mill (500). The reactions in Module 4.0 generates the desired levulinic ester (404) along with water (403) which may be recycled back to the host manufacturing site (500) and reused.

It will be understood that FIG. 1 merely shows one exemplary embodiment of the present disclosure, and one skilled in the arts readily recognizes that other process modifications may be made without departing from the concept presently disclosed.

### Module 1.0: Feed Preparation Module

In the Feed Preparation Module (Module 1.0), biomass is converted to fiber pulps. In one exemplary embodiment of this module, the biomass may be reduced in size and converted to fiber pulps having an equivalent diameter of less than approximately 0.25 inches to facilitate the subsequent hydrolysis reaction in Module 2.0. It is believed that the smaller dimensions of fiber pulps could improve the chemical efficiency of hydrolysis reaction, leading to higher product yields and more efficient operations. In the current arts on the process for producing levulinic acid-based compound from biomass, the size of biomass is reduced prior to hydrolysis by atmospheric grinding using commercially available mechanical grinding equipment. This known technique of reducing the biomass size is, however, energy-intensive and adds significant cost to the manufacturing process. In the present disclosure, the reduction in size of biomass prior to hydrolysis reaction is performed using a high-temperature refining (HTR) technique. This disclosed method of reducing biomass size consumes substantially lower energy compared to the atmospheric mechanical grinding method of known arts. A reduction of energy consumption in a range of 50% to 80% may be achieved. Additionally, the disclosed HTR treatment may defiberize biomass into individual fibers that are separated substantially at the middle lamella, which leads to a higher rate of the subsequent hydrolysis reaction.

In one embodiment of the present disclosure, the high-temperature refining of biomass comprises steps of:
(a) chipping the biomass into a predetermined size;
(b) streaming the chipped biomass at a temperature above the softening point of lignin for a predetermined time;
(c) defibering the streamed biomass to provide fiber pulps; and
(d) discharging the fiber pulps to further biomass processing steps.

In one embodiment of the present disclosure, the high-temperature refining of biomass comprises steps of:
(a) chipping the biomass into a size suitable for transporting into the mechanical refiner;
(b) conveying the chipped biomass through a pressurized steaming device that is heated to a temperature above the softening point of lignin;
(c) streaming the resulting biomass for a predetermined time;
(d) defibering the streamed biomass to provide fiber pulps;
(e) discharging the fiber pulps to further processing steps.

The temperatures of the pressurized streaming device in (b) may be in a range of about 160° C. to 175° C. However, a higher temperature may be used when desired. The chipped biomass may be steamed from about 4 minutes to about 15 minutes.

In one embodiment, the defibering step (d) may be performed using a pressurized refiner. When desired, chemicals may be added into the system during defibering process to affect the properties of the biomass, to further affect the defibering performance of the refiner, or both.

Volatile organic materials in the biomass, such as turpentine and methanol, vaporize during the HTR treatment. In one embodiment, the high-temperature refining of biomass further comprises a step of removing volatile organic materials, such as turpentine or methanol, from the fiber pulps prior to subjecting the pulps to hydrolysis reaction. The recovered volatiles may be purified and sold or utilized as high-value chemicals. These volatiles are not exposed to hydrolytic catalysts in the subsequent hydrolysis such as sulfuric acid; therefore, they contain no organo-sulfur impurities which could be difficult to remove and reduce the value of the obtained turpentine and methanol.

When desired, the generated fiber pulps may be discharged into a pressurized tank in order to retain heat energy and pressure for a subsequent pump conveyance to the hydrolysis reaction vessel of the Hydrolysis Reaction Module (Module 2.0). In this manner, the refiner may be used as a means for defibering the biomass, as well as a mean for preheating and pumping device. In one embodiment, the fiber pulps generated in the Feed Preparation Module (Module 1.0) is pre-heated prior to conveyance to the Hydrolysis Reaction Module (Module 2.0) to further improve the operability and stability of the process.

A variety of biomass may be used in the present disclosure. These include, but not limited to, sludges from paper manufacture such as waste paper sludge, ground wood sludge, hardwood paper sludge, and recycled paper sludge; agricultural residues such as corn husks, corn cobs, rice hulls, straw, bagasse, starch from corn, wheat oats, and barley; bagasse pity; bagasse; molasses; aqueous oak wood extracts; rice hull; oats residues; wood sugar slops; fir sawdust; naphtha; corncob furfural residue; cotton balls; raw wood flour; rice; straw; soybean skin; soybean oil residue; corn husks; cotton stems; cottonseed hulls; starch; potatoes; sweet potatoes; lactose; sunflower seed husks; sugar; corn syrup; hemp; waste paper; wastepaper fibers; sawdust; wood such as ground wood and hard wood; residue from agriculture or forestry; organic component of municipal and industrial wastes; waste plant materials from hard wood or beech bark; fiberboard industry waste water; post-fermentation liquor; furfural still residues; and combinations thereof.

### Module 2.0: Hydrolysis Reaction Module

In the two-reactor hydrolysis process disclosed in U.S. Pat. No. 5,608,105, C6 polymer components in the raw material are catalytically converted to substantially C5 levulinic acid and C1 formic acid, while C5 hemi-celluloses are converted to furfural. However, typically lignin component in the raw materials is not chemically converted. Instead, lignin in the raw material is thermo-mechanically converted to a solid char. Filtration or centrifugation is typically used to separate the desired levulinic acid from the char by-products. Therefore, this process relies on the particle formation at atmospheric temperature and filtration to effect the char separation. As such, the efficiency of separation may be low.

In the Hydrolysis Reaction Module of the present disclosure (Module 2.0), the char is isolated from the hydrolyzed mixture stream at a high temperature and pressurized conditions. Under these conditions, the chars exist as sticky particles, and thereby enhancing the efficiency of their separation from the reaction mixture stream. Since the recovered chars are a useful solid fuel, they can be used as energy sources for the production of levulinic acid from biomass, as well as for the host manufacturing facility itself. For example, in one exemplary embodiment wherein the disclosed process for producing levulinic acid is integrated into a pulp mill host, the recovered chars may be used to displace fossil fuels for energy sources such as in lime kilns to substantially reduce energy costs of the paper mill host and decrease the mill's carbon footprint associated with fossil fuel use.

The hydrolysis reaction suitable for use in the disclosed Module 2.0 may include acid-catalyzed or enzyme-catalyzed hydrolysis. Various acid catalysts are suitable for use in the hydrolysis module of the present disclosure. These include, but are not limited to, inorganic acids such as sulfuric acid, fluorosulfonic acid, hydrochloric acid, nitric acid, phosphoric acid, benzenesulfonic acid, phosphotungtstic acid, phosphomolybdic acid, trifluromethanesulfonic acid, 1,1,2,2-tetrafluroethanesulfonic acid, 1,2,3,2,3,3-hexapropanesulfonic acid, and mixtures thereof; organic acids such as p-toluene sulfonic acid; acidic-ion exchange resins; Brönsted acid catalysts such as bismuth triflate, yttrium triflate, ytterbium triflate, neodymium triflate, lanthanum triflate, scandium triflate, zirconium triflate, and Zn(BF₄)₂; fluorinated sulfonic acid polymers; metal salts of acid such as metal sulfonates, metal sulfates, metal trifluoroacetates, metal triflates; heteropolyacids; and perfluoroalkylsulfonic acids.

### Module 3.0: Product Separation and Recovery

The disclosed Module 3.0 includes a solvent extraction to separate the desired product from the hydrolysis product mixture.

In one embodiment of the present disclosure, furfural is used as an extracting solvent. Since furfural is itself one of the hydrolysis products, a large recycle stream and the necessity of disposing of large quantities of precipitated sodium sulfate crystals that are demanded in the process of U.S. Pat. No. 5,608,105 may be eliminated. Furthermore, unlike the process of U.S. Pat. No. 7,153,996 wherein olefin is required as an additional chemical to facilitate the product separation, the disclosed Module 3.0 uses furfural generated in the hydrolysis reaction itself to perform the product separation step. In this exemplary embodiment of Module 3.0 wherein furfural is used as extracting solvent, the reactive extraction of levulinic acid from the hydrolysis product mixture may be achieved without the need for additional chemicals.

When desired, Module 3.0 may include a distillation step after solvent extraction to further purify the separated levulinic acid, formic acid, and furfural products.

### Module 4.0: Derivative Preparation Module

In one exemplary embodiment, the disclosed process includes Derivative Preparation Module (Module 4.0) and the produced levulinic acid is converted to levulinate ester. For fuel additive application, the levulinate ester must be substantially free of levulinic acid because levulinic acid is corrosive to the fuel container and engine parts.

The diesel fuels for on-road diesel applications must contain no more than 15 ppm of sulfur element. In the exemplary embodiment wherein furfural is used as an extracting solvent in Module 3.0, furfural extraction facilitates a low-level of sulfur impurities in the downstream levulinic acid and esters.

In one embodiment of the disclosed Module 4.0, levulinate ester was prepared from levulinic acid by a transesterification process comprising steps of:
(a) esterification of levulinic acid with glycerol to produce a glycerol-levulinate ester; and
(b) transesterification of the glycerol-levulinate ester with mono-alcohol.

The esterification of levulinic acid with glycerol in step (a) may be performed in absence of low molecular weight mono-alcohol such as methanol or ethanol; therefore, the generated water by-product may be effectively removed through a simple and low-energy consuming method, such as vaporization. Additionally, the esterification step (a) does not require a complete esterification of the glycerol; a partial esterification to produce a mixture of mono- and di-esters is sufficient. Glycerol has a much higher boiling point; therefore, the unreacted glycerol in step (a) may be recovered via a partial condenser and reused. The transesterification of glycerol levulinate ester with alcohol in step (b) produces a low-boiling levulinate monoester. Both steps (a) and (b) are relatively rapid liquid-phase reactions and may be performed at relatively lower temperature and pressure compared to the known process of production levulinate ester.

Alcohols suitable for use in the present disclosure may be alkyl alcohols, alkenyl alcohols, cycloalkyl alcohols, or combinations thereof. In one embodiment of the present disclosure, the alcohols include C1-C15 alkyl alcohols.

When desired, the esterification and/or transesterification may be performed in the presence of acid catalyst. Suitable acid catalysts for use in the present disclosure may include, but are not limited to, inorganic acids, organic acid, ion exchange resins, fluorinated sulfonic acid polymers, metal salts of acids, heterogeneous acid catalysts, solid acid, Brönsted acids, and combinations thereof. Examples of inorganic acids may include, but are not limited to, sulfuric acid, phosphoric acid, hydrochloric acid, nitric acid, and mixtures thereof. Examples of organic acids may include, but are not limited to, organic sulfonic acid, triflic acid, trifluoroacetic acid, methanesulfonic acid, heteropolyacid, perfluoroalkylsulfonic acid, and combinations thereof. Examples of metal salts of acids may include, but are not limited to, metal sulfonates, metal sulfates, metal trifluoroacetates, metal triflates, and mixtures thereof. Examples of solid acids may include, but are not limited to, zeolites, fluorinated alumina, acid-treated silica, acid treated silica-alumina, acid treated clays, heterogeneous heteropolyacids, sulfated zirconia, acid-treated titania, acid-treated zirconia, heteropolyacids supported on zirconia, heteropolyacids supported on titania, heteropolyacids supported on alumina, heteropolyacids supported on silica, and combinations thereof.

In one embodiment of the present disclosure wherein the host plant includes a chemical pulp mill, the methanol generated from the chemical pulping process may be used as mono-alcohol for the trans-esterification reaction (b).

The process of producing levulinic-acid or ester from biomass of the present disclosure has several advantages over the existing process. These include, but are not limited to:
increased reaction rates attributed to the feed preparation in Module 1;
sufficient energy efficiency;
enhanced operating stability;
recovery of high value by-products such as turpentine and methanol;
faster reaction rates and less side reactions by decreasing the mass-transfer reaction rates in the hydrolysis of cellulose;
more effective recovery of levulinic acid, formic acid and furfural without reaction mix recycle;
lower costs for the separation of levulinic acid from hydrolysis product mixture attributed to the use of by-product furfural as extracting solvent in Module 3.0;
more efficient removal of char from the reaction mix;
minimum generation of waste sodium sulfate;
higher purity levulinic acid, formic acid and furfural recovered from the reaction mix;
lower capital equipment for esterification;
levulinate ester product suitable as fuel additive that provides fuel with performances in compliance with the governmental fuel specifications;
lower energy costs and carbon footprint for the host mill; and
reduced fresh water usage by host mill

While the disclosure has been described by reference to various specific embodiments, it should be understood that numerous changes may be made within the spirit and scope of the inventive concepts described. It is intended that the disclosure not be limited to the described embodiments, but will have full scope defined by the language of the following claims

1. A process of producing levulinic acid-based compounds, comprising:
(i) a feed preparation module characterized by subjecting biomass to a high-temperature refining treatment to generate fiber pulps;
(ii) a hydrolysis reaction module wherein the generated fiber pulps are hydrolyzed to provide a hydrolysis mixture including levulinic acid; and
(iii) a product separation and recovery module comprising a solvent extraction process to isolate the levulinic acid from the hydrolysis mixture.

2. The process of claim 1, further comprising a derivative preparation module characterized by conversion of the levulinic acid to derivative, the derivative including a member selected from a group consisting of ester, amide, acid anhydride, alcohol, and salt.

3. The process of claim 1, characterized by integration into an existing manufacturing facility.

4. The process of claim 3, wherein the existing manufacturing facility includes a member selected from the group consisting of pulp mills, ethanol manufacturing plants, biodiesel manufacturing plants, recycle plants, utilities plants with cogeneration, and combinations thereof.

5. The process of claim 1, wherein the high-temperature refining treatment comprises steps of:
(a) chipping the biomass into a predetermined size;
(b) streaming the chipped biomass at a temperature above a softening point of lignin for a predetermined time;
(c) defibering the streamed biomass to provide a refining mixture including fiber pulps and volatile organic materials; and
(d) discharging the refining mixture to the hydrolysis reaction module.

6. The process of claim 5, wherein the streaming temperature is in a range of about 160° C. to about 175° C.

7. The process of claim 5, wherein the defibering step in 5(c) is performed using a pressurized refiner.

8. The process of claim 5, further including a step of isolating the volatile organic materials from the refining mixture of 5(c) prior to discharging in the step 5(d).

9. The process of claim 1, wherein the biomass includes a material selected from a group consisting of sludges from paper manufacturing process; agricultural residues; bagasse pity; bagasse; molasses; aqueous oak wood extracts; rice hull;
oats residues; wood sugar slops; fir sawdust; naphtha; corncob furfural residue;
cotton balls; raw wood flour; rice; straw; soybean skin; soybean oil residue; corn husks; cotton stems; cottonseed hulls; starch; potatoes; sweet potatoes; lactose;
sunflower seed husks; sugar; corn syrup; hemp; waste paper; wastepaper fibers;
sawdust; wood; residue from agriculture or forestry; organic components of municipal and industrial wastes; waste plant materials from hard wood or beech bark; fiberboard industry waste water; post-fermentation liquor; furfural still residues; and combinations thereof.

10. The process of claim 1, wherein the hydrolysis mixture includes a solid char by-product, the char being removed from the hydrolysis reaction module under elevated temperature and pressurized conditions.

11. The process of claim 1, wherein the hydrolysis reaction module includes an enzymatic hydrolysis reaction.

12. The process of claim 1, wherein the hydrolysis reaction module include a hydrolysis reaction in a presence of acid.

13. The process of claim 12, wherein the acid includes a chemical selected from a group consisting of inorganic acids, organic acids, acidic-ion exchange resins, Brönsted acids, fluorinated sulfonic acid polymers, metal salts of acid, heteropolyacids, perfluoroalkylsulfonic acids, and combinations thereof.

14. The process of claim 1, wherein furfural is used as an extracting solvent for the solvent extraction in the product separation and recovery module.

15. The process of claim 1, wherein the product separation and recovery module further includes a distillation step.

16. A process of producing levulinic acid-based compounds, comprising:
(i) a feed preparation module characterized by subjecting biomass to a high-temperature refining treatment to generate fiber pulps;
(ii) a hydrolysis reaction module wherein the generated fiber pulps are hydrolyzed to provide a hydrolysis mixture including levulinic acid;
(iii) a product separation and recovery module including a solvent extraction process to isolate the levulinic acid from the hydrolysis mixture; and
(iv) a derivative preparation module including steps of:
(a) esterification of the levulinic acid with glycerol to produce a glycerol levulinate; and
(b) transesterification of the glycerol levulinate with mono-alcohol to produce levulinate ester of mono-alcohol.

17. The process of claim 16, wherein the mono-alcohol comprises an alcohol having a number of carbon atoms in a range of 1 carbon to 15 carbons.

18. The process of claim 16, wherein the esterification in 16(iv)(a) is performed in a presence of an acid catalyst.

19. The process of claim 16, wherein the transesterification in 16(iv)(b) is performed in a presence of acid catalyst.

20. The process of claim 16, characterized by integration into an existing manufacturing facility.

21. A levulinic acid produced from the process of claim 1.

22. A levulinic acid-based compound derived from the levulinic acid of claim 21.

23. A levulinate ester produced from the process of claim 16.

24. A fuel additive, including the levulinate ester of claim 23.

25. A fuel composition, including the fuel additive of claim 24.
